(19) United States
(12) Patent Application Publication
(10) Pub. No.: US 2013/0197630 A1
(43) Pub. Date: Aug. 1, 2013

(54) Device for Replacing at Least One Chord in The Mitral Valve, Kit Comprising at Least Two Devices, and Surgical Method

(71) Applicant: CENTRE HOSPITALIER UNIVERSITAIRE DE CLERMONT-FD, Clermont Ferrand Cedex (FR)

(72) Inventor: Kasra AZARNOUSH, Romagnat (FR)

(73) Assignee: CENTRE HOSPITALIER UNIVERSITAIRE DE CLERMONT-FD, Clermont Ferrand Cedex (FR)

(21) Appl. No.: 13/751,224

(22) Filed: Jan. 28, 2013

(30) Foreign Application Priority Data
Jan. 26, 2012 (FR) 1250757

(51) Int. Cl. A61F 2/24

(52) U.S. Cl. CPC A61F 2/2403
USPC 623/2.17

(57) ABSTRACT

The replacement device according to the invention makes it possible to replace at least one natural chord (19) making up the mitral valve, said natural chord normally being connected by one end to a pillar (20) formed by a muscular relief on the cardiac wall and, by another end, to a component leaflet (14) of the valve. The replacement device comprises a base (31) that can be fastened on a pillar (20) and on which at least one pair (P3) of chords (34) with predefined lengths are mounted that are suitable for being fastened on the leaflet (14) of the valve. Each pair (P3) of chords is configured in a loop provided with an end reinforcement for fastening on the leaflet (14) of the valve (12) opposite the base (31). The invention also relates to a kit comprising at least two replacement devices.

The invention relates to a device for replacing at least one chord of the mitral valve, as well as a kit comprising at least two devices. The invention also relates to a surgical method for replacing at least one chord of the mitral valve.

The mitral valve is a cardiac valve that separates the left auricle from the left ventricle. This valve comprises three elements:
Two valvular leaflets made up of an anterior leaflet or large mitral valve and a posterior leaflet or small mitral valve. The leaflets are fixed on one side of a fibrous ring called the mitral ring.
Two muscular pillars coming from the wall of the left ventricle and oriented toward the leaflets.
Two elastic bands of fibrous tissues, or chords connecting the free end or head of the pillars to the edges of the anterior and posterior leaflets. A distinction is made between primary or secondary chords according to the fastening area of the chords on the leaflets.

A mitral ring is arranged between the left auricle and the left ventricle and defines the passage between those two organs. The two leaflets, depending on their respective position, do or do not authorize the passage of blood between the left auricle and the left ventricle. The leaflets are set in motion by the blood circulation, the movements of the leaflets being limited by chords connecting the leaflets to the pillars formed by muscular reliefs on the cardiac wall.

When a mitral insufficiency is observed, i.e., generally, when a lack of coaptation of two leaflets is observed during ventricular systole, this causes a more or less significant reflux of the blood from the left ventricle toward the left auricle due to the fact that the two leaflets are not in sealed contact. The blood thus driven back follows the opposite path from the physiological direction. When this mitral insufficiency is severe, it causes complications such as bacterial endocarditis, expansion of the cardiac cavities, atrial or ventricular rhythm difficulties, thromboembolic episodes, etc. The recommended treatment is a surgical treatment.

It is currently preferred to reconstruct the valve rather than completely replacing it with a prosthesis. In fact, the available data shows that not only does this conservative treatment, also called mitral plasty, make it possible to preserve the ventricular geometry as well as enabling complete restoration of the function of the mitral valve, but additionally, the postoperative mortality rate is 2 to 3 times lower than in cases of replacement with a prosthesis.

This reconstruction technique is based on the fact that the malfunction of the valve is generally due to a deficiency in the chords. More specifically, the chords are broken or distended.

Mitral plasty consists of replacing the defective chords with threads or chords made from a neutral material that is biocompatible, resistant over time, and does not experience variations in its mechanical behavior, in particular elasticity. These chords are generally made from polytetrafluoroethylene or PTFE.

These threads are for example placed using minimally invasive techniques allowing access to the left auricle and the mitral valve while avoiding a sternotomy and a large incision in the rib cage. When these new chords are used, they are fastened, for example by tying or ligature, on the one hand in the leaflets, and on the other hand in the pillars.

One of the important points when placing the chords is the length of the replacement chord. This length must correspond to the length of the initial chord. An excessively short chord does not allow optimal opening of the valve, and an excessively long chord conversely does not allow effective coaptation.

These chords being placed one by one, the useful length of the chord should be determined, in reference to the available space between the leaflets and the pillars and relative to the initial natural chord. This determination is made before the placement of each replacement chord, with the understanding that, for safety reasons, two PTFE chords are preferably used to replace one natural chord.

In other words, the placement of the chords is not easy, since it involves periodically and repetitively measuring to determine the length of each chord, which involves a relatively lengthy operation. However, the cardiac arrest under artificial blood shunt done during this operation must be as brief as possible.

US-A-2010 179 574 describes a device for replacing at least one natural chord making up the mitral or tricuspid valve. The device comprises a base suitable for being fastened on a pillar and on which a single pair of chords is mounted adapt to be fastened on a leaflet of the valve by their free ends. The two chords can slide relative to the base and have an adjustable length. Alternatively, the two chords can be fastened to the base and have a predefined length. However, such a device is suitable for replacing a single broken or defective chord. Due to the bulk of the base, it is difficult, if not impossible, to fasten several bases to a same muscular pillar to use several devices in parallel.

The invention more particularly aims to resolve the various drawbacks mentioned above by proposing a device for replacing chords of the mitral valve whereof the working length is easy to determine, making it possible to replace several chords in parallel if needed, and allowing rapid placement making it possible to decrease the clamping time, i.e., to decrease the duration of the cardiac arrest under artificial blood shunt.

To that end, the invention relates to a replacement device for replacing at least one natural chord making up the mitral valve, said natural chord normally being connected by one end to a pillar formed by a muscular relief on the cardiac wall and, by another end, to a component leaflet of the valve. This replacement device comprises a base that can be fastened on a pillar and on which at least two pairs of chords with predefined lengths are mounted that are suitable for being fastened on the leaflet of the valve by their ends opposite the base. Each pair of chords is configured in a loop provided with an end reinforcement for fastening on the leaflet of the valve opposite the base.

The fastening of such a device is quick, since the chords are matched two by two forming a loop provided with end reinforcements, and the base of the device need only be fastened on the pillar and the end reinforcements on the leaflet, and no longer each chord.

According to advantageous optional aspects of the invention, such a replacement device may incorporate one or more of the following features:
For each pair, the ends of the chords opposite the base are attached to each other forming the loop, and are free relative to the ends of the chords of the other pairs.
Each end reinforcement is fastened on the loop formed by the pair of chords.
Each end reinforcement slides on the loop formed by the pair of chords.
The end reinforcements are made from a material similar to the fastening base on the pillar.
The end reinforcements are provided with at least one insertion thread provided with a piercing element for fastening on a leaflet.
The base and the chords are a single piece, made from a same material.
The base is provided with at least one through opening allowing it to be fastened on a pillar.
The base is provided with at least one insertion thread provided with a piercing element for fastening to the pillar.
The device comprises a fastening plate gripping the pillar as a complement to the base.
The device comprises three pairs of chords.
The various chords fastened on a same base have the same length.
The various chords fastened on a same base have different lengths.
The chords are made from polytetrafluoroethylene.
The chords have lengths from 5 to 35 mm.
The base is configured in a square or rectangle.

The invention also relates to a kit for replacing at least one chord of the mitral valve, wherein the kit comprises at least two replacement devices according to at least one of the previous features.

According to advantageous but optional aspects, such kits may incorporate at least one of the following features:
Each replacement device is adapted to a predefined length of the natural chords to be replaced.
Each chord length is identified by a distinctive sign, for example a color, an icon or a number.
The kit is sterile and used only once.
The kit comprises a measurement tool suitable for measuring the length of the natural chords, for example a bent and graduated rod as currently used.
At least some replacement devices comprise a different number of chords from one another.

The invention also relates to a mitral surgery method using a device according to one of the previous features. In other words, the invention also relates to a surgical method for replacing at least one natural chord making up the mitral valve, said natural chord being connected by one end to a pillar formed by a muscular relief on the cardiac wall, and by another end to a component leaflet of the mitral valve. The method comprises at least the following steps:
a step for identifying the broken or defective natural chord(s) making up the mitral valve;
a step for measuring the working length of the natural chord(s) to be replaced, in particular using a suitable measuring tool;
a step for selecting a replacement device, comprising a base that can be fastened on a pillar and at least two pairs of chords whereof the number and length are selected by the surgeon as a function of the natural chord(s) to be replaced, within a surgical kit comprising at least two different replacement devices, each chord pair being configured in a loop and provided with an end reinforcement for fastening on the leaflet of the valve opposite the base;
a step for placing the replacement device on the mitral valve, with artificial blood shunt cardiac circulation, consisting of fastening the base on a pillar, for example by tying or ligature, and the end reinforcements of the chords on the leaflet.

Optionally, when useful, the method also comprises a step for removing certain natural chord(s), carried out between the identification step and the selection and placement step.

The invention will be better understood, and other advantages thereof will appear more clearly, upon reading the following description of a device for replacing chords of the mitral valve according to several embodiments of the invention and a replacement kit, provided solely as an example and done in reference to the appended drawings, in which:

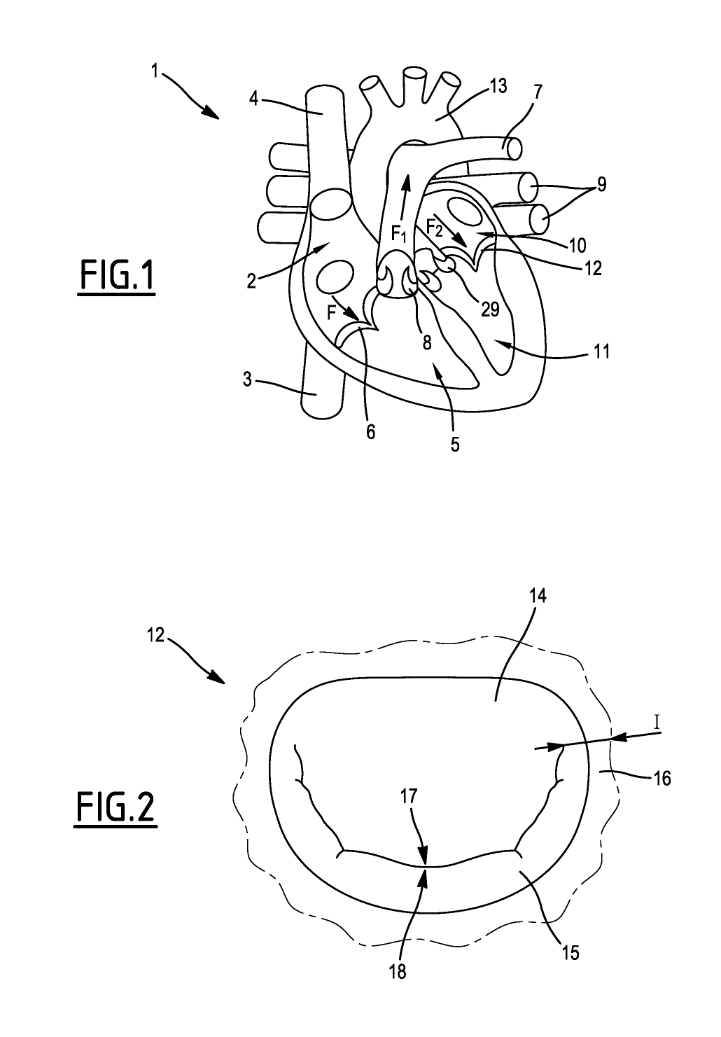

FIG. 1 is a partial cross-section of a heart, the left and right auricles, the left and right ventricles, the arteries, veins, and the various valves being shown diagrammatically,

FIG. 2 is an enlarged simplified top view of only the mitral valve in the closed configuration,

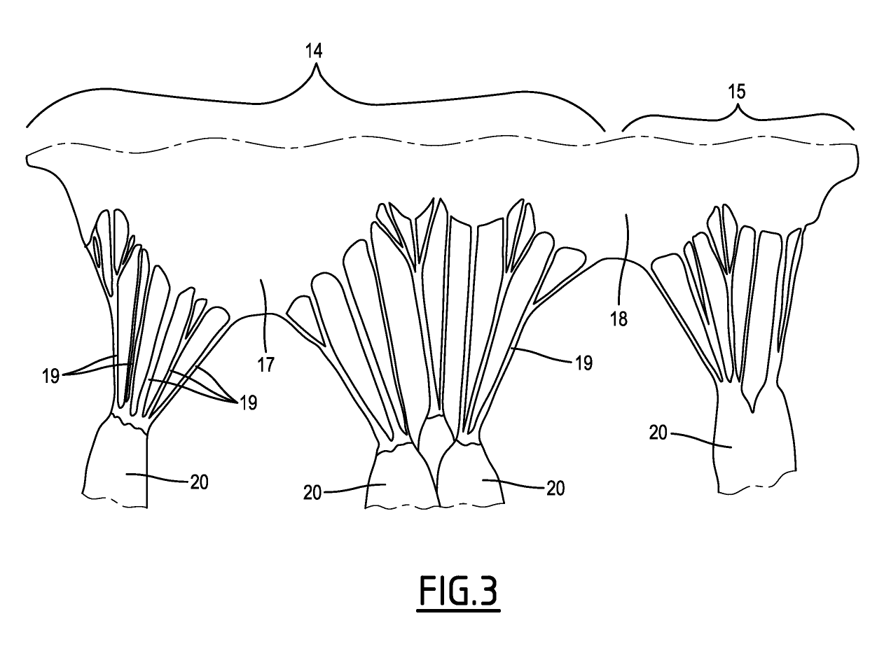

FIG. 3 is an enlarged simplified side view of the pillars, the chords, and two component leaflets of a mitral valve in an unfolded configuration, i.e., after sectioning the mitral ring,

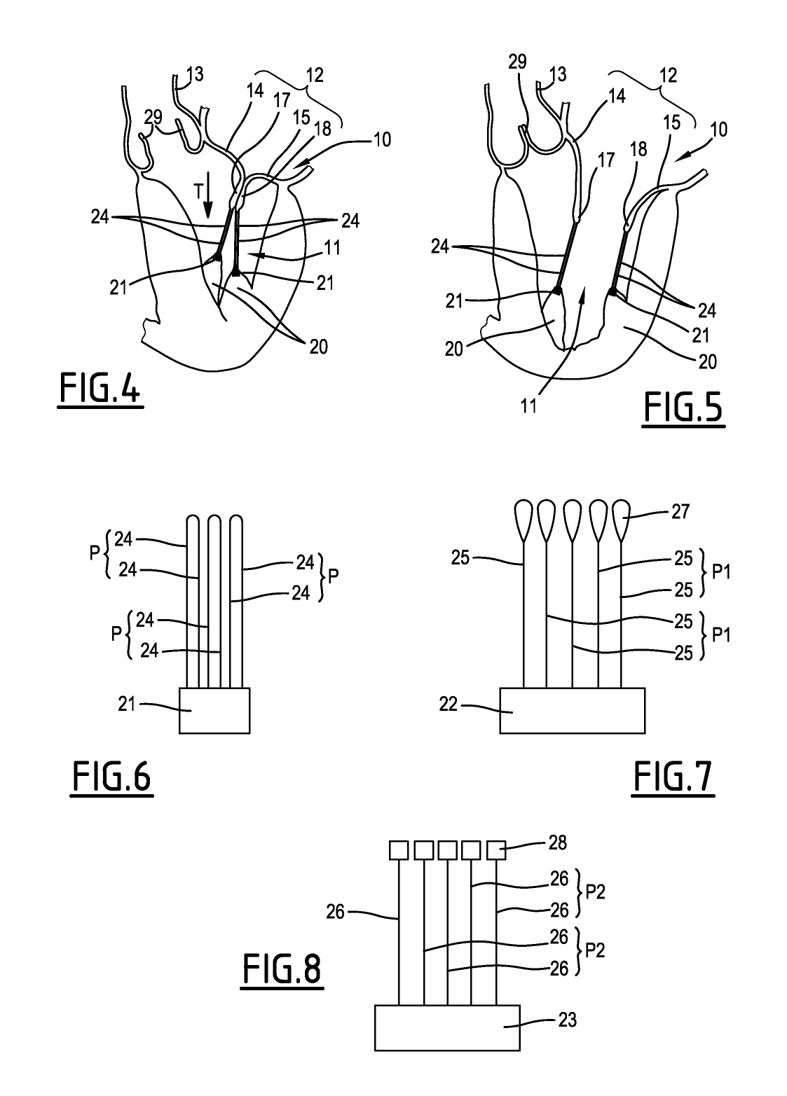

FIGS. 4 and 5 are operating diagrams of the mitral valve showing part of the left ventricle, the auricle of the left atrium and the aorta in ventricular systole in FIG. 4 and ventricular diastole in FIG. 5, a single chord fastened on a pillar being illustrated,

FIGS. 6 to 8 are simplified front views on another scale, illustrating three embodiments of a replacement device,

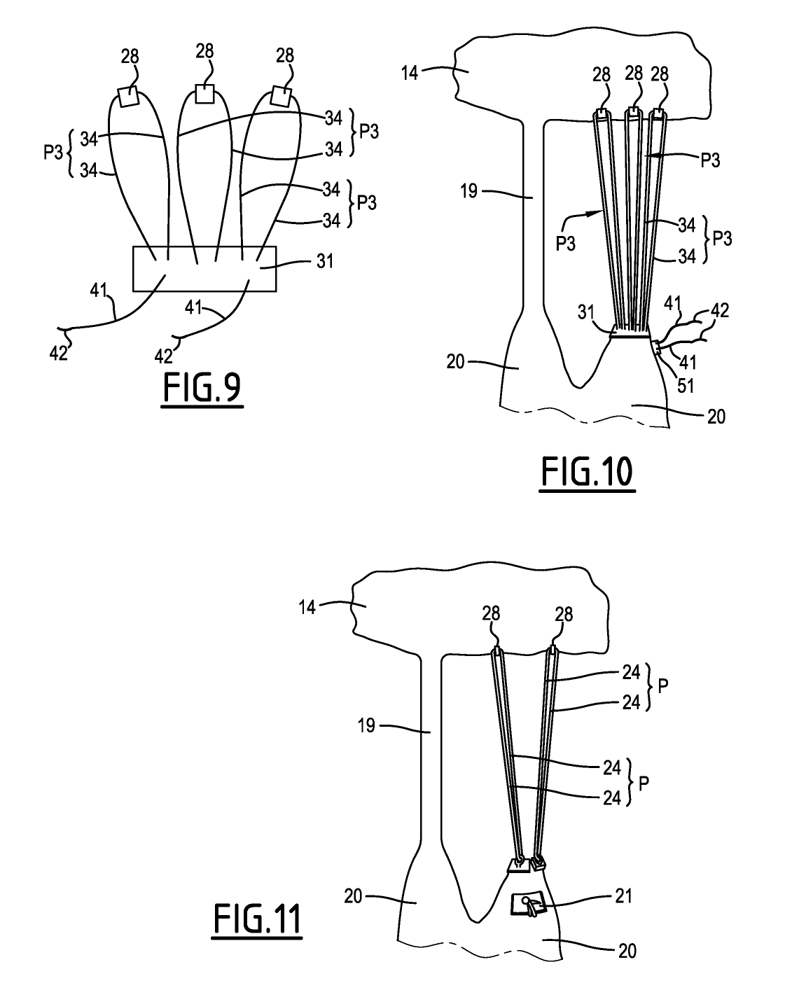

FIG. 9 is a simplified front view, similar to those of FIGS. 6 to 8, illustrating the preferred embodiment of the replacement device according to the invention,

FIG. 10 is a simplified front view of the device of FIG. 9, on another scale, partially equipping a mitral valve, and

FIG. 11 is a simplified front view, on another scale, of the device of FIG. 6 provided with end reinforcements, partially equipping a mitral valve.

FIG. 1 shows a diagram part 1, with the understanding that this may be the heart of a human being or an animal, for example a mammal. In other words, the invention that will be described hereafter applies both in human and veterinary medicine, irrespective of the age of the patient.

The oxygen-depleted blood enters the right auricle 2 through the inferior 3 and superior 4 vena cava. It goes into the right ventricle 5 during a systole/diastole alternation of the right auricle 2 and ventricle 5 following arrow F. A non-rebreathing valve, the tricuspid valve 6 prevents the reflux of blood from the right ventricle 5 to the right auricle 2. During a systole, i.e., a contraction of the right ventricle 5, the blood is sent by means of the pulmonary artery 7, along the arrow F1, into the lungs to be refreshed. The pulmonary valve 8 prevents the reflux of blood into the right ventricle 5.

A similar operation is observed in the left part of the heart. The oxygenated blood coming from the lungs to the pulmonary veins 9 enters the left auricle 10 which, during its systole, sends the blood into the left ventricle 11 along the arrow F2. During the ventricular systole, the mitral valve 12, like the tricuspid 6 and pulmonary 8 valves, forms a non-rebreathing valve preventing the oxygenated blood that is returned to the body through the aorta 13 from flowing back into the left auricle 10.

As diagrammatically shown in FIGS. 1, 2, 4 and 5, the mitral valve 12 comprises two leaflets 14, 15 suitable either for moving away from one another and allowing the passage between the left auricle 10 and ventricle 11, or, as illustrated in FIG. 2, being in coaptation, i.e., in sealed mutual contact and thereby closing off the passage between the left auricle 10 and ventricle 11.

FIG. 2 illustrates a mitral valve 12 with the anterior leaflet 14 situated toward the top, relative to FIG. 2, and the posterior leaflet 15 situated toward the bottom. These leaflets 14 and 15 have different configurations and sizes. Due to its length, the posterior leaflet 15 generally extends over a little more than half of the circumference of the rings 16 defining the working passage of the mitral valve 12. Said ring 16 has a substantially circular shape. The leaflet 15 has a smaller width than the width of the anterior leaflet 14. The anterior leaflet 14 is also more flexible, and its movement during operation is greater than that of the posterior leaflet 15. In other words, the posterior leaflet 15 forms a flexible support for the anterior leaflet 14 during movements of the latter part.

If an incision I is made in the ring 16 up to the posterior leaflet 15, at one end of the coaptation area, it is possible to unfold the valve to obtain a view similar to that shown in FIG. 3. The anterior leaflet 14 is on the left in FIG. 3.

The part 17 of the anterior leaflet 14 suitable for sealably bearing against the part 18 of the posterior leaflet 15 is oriented toward the bottom left in FIG. 3. It will be noted that the edges of the parts 17 and 18 both of the posterior 15 and anterior 14 leaflets are provided with chords 19. Said chords are formed by elongated elements connecting the end of the leaflets 14, 15 to areas situated on the inner wall of the ventricle 11 in a tree structure. These areas, called pillars 20, are muscular reliefs situated on the inner surface of the wall of the heart, in this case the ventricular wall. In other words, the chords 19 connect a moving part, i.e. the anterior 14 and posterior 15 leaflets, to a fixed part, i.e., the pillars 20, like the ropes of a parachute.

When a mitral insufficiency occurs, one or more chord(s) 19 is (are) damaged, or sectioned, which undermines the sealing of the coaptation of the leaflets 14 and 15. In fact, the complete absence of chords 19 does not influence the passage of blood from the auricle 10 to the ventricle 11, since that is done by separating the leaflets 14 and 15. However, a defect in the integrity of at least one chord 19 causes a modification in the return force of that chord, which does not provide optimal traction on the leaflet to which it is fastened. However, to achieve effective coaptation, there must be homogenous bearing, on a given surface, of the parts 17 and 18 on one another. In other words, maintenance of the bearing of the ends 17, 18 of the leaflets 14, 15 is not sufficient to prevent their separation due to the pressure of the blood present in the ventricle 11 and therefore causing a blood reflux in the auricle 10.

Replacing the chord(s) 19 first requires identifying the broken or defective chord(s). The removal of the chord(s) to be replaced is at the discretion of the surgeon, and in some cases, they are left in place. The following step is the placement of a replacement device. Inasmuch as this is a relatively major surgical operation with an artificial blood shunt, it is necessary to ensure that the replacement device will not only preserve its integrity, but that it will also preserve its mechanical features, in particular its elasticity, over time, given that the systole/diastole alternation occurs 80 times per minute on average. In other words, the replacement device must not contract and stretch over time, its elastic properties, and therefore its return force, remaining constant.

The replacement device, illustrated according to different alternatives in FIGS. 6 to 9, is made from a neutral biocompatible material with time-stable and resistant mechanical properties. Advantageously, it is made from polytetrafluoroethylene (PTFE).

The device comprises a base 21, 22, 23, 31 as illustrated in FIGS. 6 to 11. This base is configured in a square 21 or rectangular 22, 23, 31 plate. This plate 21, 22, 23, 31 has dimensions suitable for being mounted on a pillar 20. For example, a square base 21 is between 1 and 10 mm per side and a rectangular base 22, 23, 31 is from 2 to 10 mm by 2 to 10 mm. This plate 21, 22, 23, 31 has predefined mechanical properties, in particular a given rigidity. Advantageously, the plate 21, 22, 23, 31 of the various embodiments may be provided with at least one opening, not shown, for fastening thereof on the pillar 20.

Artificial chords 24, 25, 26, 34 are fastened on this base. In order to ensure optimal safety and offset any defect in the device, there is a redundancy of the chords 24, 25, 26, 34 on the base 21, 22, 23, 31. It is therefore chosen to double the replacement chord or even, in some cases where necessary, to triple it according to the chord length and/or the patient.

In the invention, at least two pairs of chords 24, 25, 26, 34 are arranged on a base 21, 22, 23, 31. Preferably, as illustrated in FIGS. 6 to 9, either three pairs P or P3, or two pairs P1, P2 of chords 24, 34, 25, 26, respectively, are used. In the different illustrated embodiments, the chords 24, 25, 26, 34 have one end fastened on their base 21, 22, 23, 31, respectively.

FIG. 6 illustrates a first embodiment in which the device comprises three pairs P of chords 24, where each pair P is formed in a loop. Each pair P can comprise a single thread folded to form the loop including the two chords 24. One end of each chord 24 is fastened on the base 21 using known techniques. The other ends of the chords 24 are attached, within each pair P, such that the loop is closed opposite the base 21. The three pairs P have the same length.

FIG. 7 illustrates another embodiment, where a base 22 with larger dimensions comprises two pairs P1 of chords 25 and one single chord 25. The free upper end of each chord 25 is configured in a loop 27. Such an embodiment is suitable for cases where one must use chords 25 with a large diameter.

FIG. 8 illustrates another embodiment, similar to FIG. 7, i.e., with two pairs P2 of chords 26 and one single chord 26. The free end of each chord 26 is no longer configured in a loop, but is instead provided with a fastening plate 28 similar to the base 23, in particular in terms of fastening material. Each of the plates 28 forms an end reinforcement for the chord 26, which facilitates the fastening thereof to the leaflet 14 or 15. Here, a device is used with chords 26 having a large diameter, whereof the fastening on the leaflets 14, 15 is done using the end reinforcements 28 similarly to the fastening of the base 23 on the pillar 20. In one alternative not illustrated, each end reinforcement 28 is provided with a through opening.

FIG. 9 illustrates the preferred embodiment of the invention, in which the device comprises three pairs P3 of chords 34, where each pair P3 is configured in a loop and is provided with an end reinforcement 28 positioned opposite the base 31. The reinforcements 28 in particular facilitate the passage of needles, and therefore the fastening of the pairs P3 of chords 34 to the leaflet 14 or 15. For each chord pair P3, the so-called free end of the chords 34 provided with the reinforcement 28 can be moved freely relative to the ends of the other pairs P3 of chords 34. The reinforcement 28 can be fastened to the pair P3 of chords 34 or, alternatively, can be arranged slidingly on the pair P3 of chords 34. In that case, the reinforcement 28 can move over the loop formed by the pair P3 of chords 34 and is positioned optimally during weight bearing of the mitral valve.

The device of FIG. 9 can also include two insertion threads 41 that are attached to the base 31 and whereof the end opposite the base 31 is provided with a needle 42, hook or other piercing element. The threads 41 and the needles 42 facilitate fastening of the base 31 to the pillar 20, as shown in FIG. 10. A reinforcing plate 51 can be arranged on one side of the pillar 20 opposite the base 31, passed through by the needles 42 then the threads 41, so as to further reinforce the anchoring of the base 31 of the device on the pillar 31. The pillar 20 is then sandwiched between the base 31 and the reinforcing plate 51 during fastening of the device on the pillar 20. In that case, the fastening on the pillar 20 is done by tying the insertion threads 41 passing through the base 31, the pillar 20 and the reinforcing plate 51. Said reinforcing plate 51 is advantageously similar to the base 31.

In FIGS. 6 to 9, the chords 24, 25, 26, 34 that are fastened on a same base 21, 22, 23, 31 have a same length. Alternatively, they have different lengths.

The chords 24, 25, 26, 34 are fastened to the bases 21, 22, 23, 31 using known techniques, i.e., by gluing using a biocompatible glue, high-frequency welding, ligature, or preferably, are a single piece with the base. They are then made from the same material as the base, for example PTFE, during the manufacture of the device, for example by injection or molding.

The length of a natural chord 19 varies, not only from one chord to another, over a same mitral valve, with the understanding that that variation depends on the pillar on which the chord 19 is attached, but also from one individual to another. In general, in an adult human, the working length of the chord 19 is estimated to be comprised between 10 and 25 mm.

The operation of the valve is illustrated in FIGS. 4 and 5. Here, a single chord 24, as illustrated in the device according to the embodiment of FIG. 6, is shown, with the understanding that this operation is identical with a natural chord 19 or with a chord of a device according to another embodiment of the invention.

In FIG. 4, a ventricular systole is shown. In this case, the pressure of the blood in the ventricle 11 is sufficient to oppose the traction T of the chords 24 and to keep the latter stressed. This blood pressure makes it possible to push back the anterior leaflet 14 and the upper leaflet 15 toward the left auricle 10 while bringing the ends 17 and 18, respectively, of the leaflets 14 and 15 closer together. The leaflets are thus in mutual sealed contact or coaptation at the parts 17 and 18 of the leaflets 14 and 15.

In this closed configuration, the mitral valve 12 has an arrow wing-shaped transverse section. In that case, the chords 24 do not oppose the coaptation movement of the leaflets 14 and 15, the return forces being lower than the blood pressure. The chords 24, due to their length selected by the surgeon and their adaptive elasticity, participate in sealing the coaptation while keeping the parts 17, 18 of the leaflets 14, 15 in a stretched position, which makes it possible to have a large coaptation surface.

Here, elasticity of the chords 24, 25, 26, 34 refers to a rigidity of the chords that is suitable, during diastole and systole movements, for not causing lesions on the pillar 20 to which they are connected or on the leaflet 14 or 15, while also ensuring sufficient tension to keep the leaflets 14, 15 in sealed contact at the areas 17, 18 when the ventricle 11 fills with blood.

In this configuration, the blood is sent into the aorta 13, the aortic valve 29 being open. Once the blood has passed from the ventricle 11 into the aorta 13, the blood pressure decreases in the ventricle 11. The auricle 10 has, during the evacuation of the blood in the aorta 13, filled with blood coming from the lungs by means of the pulmonary veins 9. The blood pressure increases in the auricle 10 significantly relative to the blood pressure in the ventricle 11. This pressure difference makes it possible to separate the two leaflets 14 and 15 from each other. This is favored by the return exerted by the chords 24 and by a relative flexibility of the pillars 20, which “accompany” this movement by moving away from each other to favor the separation of the posterior 15 and anterior 14 leaflets and to define a working passage for the blood with optimal dimensions between the auricle 10 and the ventricle 11. During this movement, the blood flows in the ventricle 11, along the arrow F2, not being hindered in its path by the leaflets 14 and 15, which free the passage, or by the chords 24, which are small. During filling of the ventricle 11, the aortic valve 29 is closed.

The proper operation of the mitral valve therefore involves using chords with a suitable and controlled length. To fasten using a technique known in itself, for example by ligature or tying, on the one hand, the free end of a chord 24, 25, 26, 34 in the anterior 14 or posterior 15 leaflet and, on the other hand, the base 21, 22, 23, 31 on a pillar, the appropriate length of the chord 24, 25, 26, 34 should be determined beforehand as a function of the natural chord 19 to be replaced.

The placement of such a device is illustrated in FIGS. 10 and 11, using a device as respectively shown in FIG. 9 and FIG. 6 provided with end reinforcements 28, with the understanding that the placement is similar for the other embodiments, for example those shown in FIGS. 7 and 8. Only the manner in which the free end of the chords is fastened on the leaflets 14, 15 varies.

In a first step, the placement of the replacement device consists of measuring the working length of the chord(s) 19 to be replaced using a known tool, for example formed by a graduated and bent rod. In a subsequent step, after having identified the defective chord(s) 19 to be replaced, the device is selected whereof the length and the number of chords 24 or 34 are suitable for the length and number of chord(s) 19 to be replaced. In a subsequent step, the base 21 or 31 is then fastened on a pillar 20, using known techniques, for example by ligature using a link passing through the opening formed in the base. The ligature is done in the thickness of the end portion of the pillar 20.

If only one chord 19 is being replaced, it is advantageously possible, as illustrated in FIG. 11, to use only two pairs P of chords 24 out of the three pairs P available on the device of FIG. 6. In that case, the unused pair P is removed, preferably once the two pairs P of chords 24 are fastened on the leaflet 14 or 15.

The fastening of the free ends of the chords 24 is also done by ligature using a link, in a known manner. The ligature is done in the thickness of the parts 17, 18 of the leaflets 14, 15.

Thus, as illustrated in FIG. 11, a pair P of chords 24, without having a bulk larger than that of a natural chord 19, offers optimal resistance and elasticity.

The choice of the length and number of the chords 24, 25, 26, 34, therefore the device, is facilitated by producing a kit comprising a set of several devices according to the invention. The devices of the kit may advantageously comprise different lengths of chords so as to cover the majority of the lengths of natural chords 19 commonly encountered in human beings or animals in the case of a kit intended for veterinary use. The kit is advantageously sterile. It is used only one time.

The kit thus comprises at least two devices, advantageously five devices, several copies of each of which may be provided, whereof the chords 24, 25, 26, 34 for example have lengths of 5 to 35 mm by variable or constant pitch. In particular, the lengths vary from 9 to 30 mm and, preferably, from 9 to 25 mm, advantageously with a constant pitch of 2 mm. In other words, in a kit, each device is adapted to a predefined length of natural chords 19 to be replaced. Advantageously, the kit comprises a measuring tool suitable for measuring the lengths of the natural chords 19, for example a bent and graduated rod as currently used.

For each defined length, the kit may also comprise devices having different numbers of chords and/or chords whereof the free ends are equipped with particular fastening means, for example those illustrated in FIGS. 7, 8 and 9. By including devices comprising different numbers of chords, the kit makes it possible to adapt quickly to the number of broken or defective natural chords 19 identified beforehand.

The identification of the different devices in a kit or different kits is facilitated using a recognizable sign, for example an icon, a number, a particular shape of the base and/or a color either of the chord or the packaging thereof.

In this way, a practitioner can easily choose the device from the kit best suited to the mitral plasty to be done and place it quickly, since it is not necessary to measure the length of each of the replacement chords each time, since they are pre-sized.

It is advantageously possible to consider devices with chords having lengths slightly longer than the length of the natural chords 19, so as to have an additional portion of the chord, serving to provide the ligature of the free end on the leaflets. In that case, the working length of the chord 24, 25, 26, 34, once positioned, is in fact that corresponding to the previously defined length, i.e., comprised between 5 and 35 mm, the raw length of the chords 24, 25, 26, 34 before they are placed being longer. In another embodiment, it is possible to have lengths of up to 50 mm, for example in veterinary surgery.

With such a device, aside from saving time when the replacement chords are placed, the risk of error is decreased, since it is no longer necessary to repetitively measure the length of the chord 19 to be replaced. This saved time also makes it possible to decrease the length of the cardiac arrest on artificial blood shunt of the patient, and therefore to decrease postoperative risks.

In one alternative that is not shown, the base is formed not by a plate made from the same material as the chords, but for example by a ligature done at the end of the chords assembled in a group or by tying the ends of the chords.

In another alternative not shown, the free end of the chords is reinforced, for example by an excess thickness of the chord or a reinforcing element, to facilitate fastening of the chords on the leaflets.

Such a replacement device can be used to repair valves other than the mitral valve, in particular to repair the chords of the tricuspid valve.

1. A replacement device for replacing at least one natural chord (19) making up the mitral valve (12), said natural chord (19) normally being connected by one end to a pillar (20) formed by a muscular relief on the cardiac wall and, by another end, to a component leaflet (14, 15) of the valve, this replacement device comprising a base (21, 22, 23, 31) that can be fastened on a pillar (20) and on which at least two pairs (P, P1, P2, P3) of chords (24, 25, 26, 34) with predefined lengths are mounted that are suitable for being fastened on the leaflet (14, 15) of the valve (12) by their ends opposite the base (21, 22, 23, 31), wherein each pair (P, P1, P2, P3) of chords is configured in a loop provided with an end reinforcement for fastening on the leaflet (14, 15) of the valve (12) opposite the base (21, 22, 23, 31).

2. The replacement device according to claim 1, wherein for each pair (P, P3), the ends of the chords (24, 34) opposite the base (21, 22, 23, 31) are attached to each other forming the loop, and are free relative to the ends of the chords (24, 34) of the other pairs (P, P3).

2. The replacement device according to claim 1, wherein each end reinforcement (28) is fastened on the loop formed by the pair (P3) of chords (34).

4. The replacement device according to claim 1, wherein each end reinforcement (28) slides on the loop formed by the pair (P3) of chords (34).

5. The replacement device according to claim 1, wherein the end reinforcements (28) are made from a material similar to the fastening base (23, 31) on the pillar (20).

6. The replacement device according to claim 1, wherein the base (21, 22, 23, 31) and the chords (24, 25, 26, 34) are a single piece, made from a same material.

7. The replacement device according to claim 1, wherein the base (21, 22, 23, 31) is provided with at least one through opening allowing it to be fastened on a pillar (20).

8. The replacement device according to claim 1, wherein the base (21, 22, 23, 31) is provided with at least one insertion thread (41) provided with a piercing element (42) for fastening to the pillar.

9. The replacement device according to claim 1, wherein a fastening plate (51) grips the pillar as a complement to the base (21, 22, 23, 31).

10. The replacement device according to claim 1, comprising three pairs (P, P3) of chords (24, 34).

11. The replacement device according to claim 1, wherein the various chords (24, 25, 26, 34) fastened on a same base (21, 22, 23, 31) have the same length.

12. The replacement device according to claim 1, wherein the various chords (24, 25, 26, 34) fastened on a same base (21, 22, 23, 31) have different lengths.

13. A kit for replacing at least one chord of the mitral valve, wherein the kit comprises at least two replacement devices according to claim 1.

14. The kit according to claim 13, wherein each replacement device is adapted to a predefined length of the natural chords (19) to be replaced.

15. The kit according to claim 13, wherein each chord (24, 25, 26, 34) length is identified by a distinctive sign, for example a color, an icon or a number.

16. The kit according to claim 13, wherein the kit is sterile and used only once.

17. The kit according to claim 13, wherein the kit comprises a measurement tool suitable for measuring the length of the natural chords (19).

18. The kit according to claim 13, wherein at least some replacement devices comprise a different number of chords (24, 25, 26, 34) from one another.

19. A surgical method for replacing at least one natural chord (19) making up the mitral valve (12), said natural chord (19) being connected by one end to a pillar (20) formed by a muscular relief on the cardiac wall, and by another end to a component leaflet (14, 15) of the mitral valve (12), the method comprising at least the following steps:
a step for identifying the broken or defective natural chord(s) (19) making up the mitral valve (12);
a step for measuring the working length of the natural chord(s) (19) to be replaced;
a step for selecting a replacement device, comprising a base (21, 22, 23, 31) that can be fastened on a pillar (20) and at least two pairs (P, P1, P2, P3) of chords (24, 25, 26, 34) whereof the number and length are selected by the surgeon as a function of the natural chord(s) (19) to be replaced, within a surgical kit comprising at least two different replacement devices, each chord pair (P, P1, P2, P3) being configured in a loop and provided with an end reinforcement (28) for fastening on the leaflet (14, 15) of the valve (12) opposite the base (21, 22, 23, 31);
a step for placing the replacement device on the mitral valve, with artificial blood shunt cardiac circulation, consisting of fastening the base (21, 22, 23, 31) on a pillar (20) and the end reinforcements (28) of the chords (24, 25, 26, 34) on the leaflet (14, 15).

20. The surgical method according to claim 19, also comprising a step for removing certain natural chord(s) (19), carried out between the identification step and the selection and placement step.
